(19) United States
(12) Patent Application Publication
(10) Pub. No.: US 2020/0138961 A1
(43) Pub. Date: May 7, 2020

(54) Method for Delivery of Medicaments via Vaporized Emu Oil Carrier

(71) Applicants: Gary Allen Lowe, Bakersfield, CA (US); Vickie Lee Lowe, Bakersfield, CA (US)

(72) Inventors: Gary Allen Lowe, Bakersfield, CA (US); Vickie Lee Lowe, Bakersfield, CA (US)

(21) Appl. No.: 16/181,047

(22) Filed: Nov. 5, 2018

(51) Int. Cl. A61K 47/46; A61M 11/04; A61K 9/00; A61K 36/185

(52) U.S. Cl. CPC A61K 47/46; A61K 36/185; A61K 9/007; A61M 11/041

(57) ABSTRACT

A method for delivery of medicaments via vaporized emu oil carrier is disclosed. A composition of emu oil and at least one medicament coagent is subjected to vaporization interior to a volume. The vapor is collected towards a single point disposed in fluid communication with the volume. The vapor is then inhaled by a patient whereby the emu oil comprising the composition speeds absorption of the at least one medicament into the pulmonary tissue to increase the effectiveness of treatment with the medicament. In an example embodiment contemplated, the at least one medicament includes cannabinoids.

### CROSS-REFERENCE TO RELATED APPLICATIONS

Not Applicable

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### BACKGROUND OF THE INVENTION

The efficacy of a drug or treatment often correlates with the speed and efficiency of the medicament's delivery to a particular targeted tissue or area in the body. Since delivery of medicaments into the body through neighboring tissue relies on diffusion, the speed of delivery of the medicament is controlled by concentration gradients, solubility, immiscibility, lipophilic and hydrophobic interaction, among other biochemical and biological processes. Increasing the effectiveness of delivering medicaments into the body is considered advantageous in the treatment of various diseases where particular medicaments are better absorbed into targeted tissue and are thereby more completely and efficiently delivered thereto.

It is established that emu oil comprises various substances, including v myristic acid, palmitic acid, palmitoleic acid, margaric acid, searic acid, elaidic acid, oleic acid, vaccenic acid, linoleic acid, linolenic acid, adachidie acid, and eicosenoic acid, which have been shown to have therapeutic, anti-inflammatory properties, and to readily pass through the epidermis, endothelial cells, and other cellular structures into underlying, neighboring, and subcutaneous tissue. Emu oil is known to be an effective carrier as a trans-cellular agent because of its remarkable interaction with human tissue. Emu oil can therefore be used as a cariier to more efficiently penetrate human tissue for the delivery of additional medicaments as coagents therewith.

It is further established in the art that various types of compositions of cannabinoids are known for use in therapeutic treatment of various disorders and discomforts, such as, for example, as an analgesic for treatment of arthralgia, neuralgia, inflammation, for inducing appetite, treatment of sleep apnea, hypertension, inhibiting growth of cancerous cells, among many other medical treatments and therapies. The pharmacological and therapeutic properties of cannabinoids are undergoing substantial discovery over the last few decades in the modern world, and are subject to a growing amount of scientific research. Cannabinoids, and their effect upon the eponymously named Endocannabinoid System in the human body, have remarkable properties in restoring the human body to health, regulating homeostasis, treating pain, inhibiting growth of cancerous cells, inducing appetite, assisting mood and sleep disorders, treating digestive disorders and other disorders and diseases. See, for example, the following, included herein by reference:
Blake et. al. 2006. Preliminary assessment of the efficacy, tolerability and safety of a cannabis medicine (Sativex) in the treatment of pain caused by rheumatoid arthritis. Rheumatology 45: 50-52.
Ware et al. 2005. The medicinal use of cannabis in the UK: results of a nationwide survey. International Journal of Clinical Practice 59: 291-295.
Malfait et al. 2000. The nonpyschoactive cannabis constituent cannabidiol is an oral anti-arthritic therapeutic in murine. Journal of the Proceedings of the National Academy of sciences 97: 9561-9566
Sumariwalla et al. 2004. A novel synthetic, nonpsychoactive cannabinoid acid (HU-320) with anti-inflammatory properites in murine collagen-induced arthritis. Arthritis & Rheumatism 50: 985-998.
Neff et al. 2002. Preliminary observation with dronabinol in patients with intractable pruritus secondary to cholestatic liver disease. American Journal of Gastroenterology 97: 2117-2119.
Dvorak et al. 2003. Histamine induced responses are attenuated by a cannabinoid receptor agonist in human skin (PDF). Inflammation Research 25: 238-245.
Dvorak et al. 2003. Cannabinoid agonists attenuate capsaicin-induced responses in human skin. Pain 102: 283-288.
Szepietowski et al. 2005. Efficacy and tolerance of the cream containing structured physiological lipid endocannabinoids in the treatment of uremic pruritus: a preliminary study. Acta Dermatovenerologic Croatica (Croatia) 13: 97-103.
Paus et al. 2006. Frontiers in pruritus research: scratching the brain for more effective itch therapy. Journal of Clinical Investigation 116: 1174-1185.
Ofek et al. 2006. Peripheral cannabinoid receptor, CB2, regulates bone mass. Proceedings of the National Academy of Sciences of the United States of America 103: 696-701.
Itia Bab. 2007. Regulation of Skeletal Remodeling by the Endocannabinoid System. Annals of the New York Academy of Sciences 1116: 414-422.
Wade et al. 2006. Long-term use of a cannabis-based medicine in the treatment of spasticity and other symptoms of multiple sclerosis. Multiple Sclerosis 12: 639-645.
Wade et al. 2003. A preliminary controlled study to determine whether whole-plant cannabis extracts can improve intractable neurogenic symptoms. Clinical Rehabilitation 17: 21-29.
Meinck et al. 1989. Effects of cannabinoids on spasticity and ataxia in multiple sclerosis. Journal of Neurology 236: 120-122.
Denis Petro. 1980. Marijuana as a therapeutic agent for muscle spasm or spasticity. Psychosomatics 21: 81-85.
Pryce et al. 2003. Cannabinoids inhibit neurodegeneration in models of multiple sclerosis. Brain 126: 2191-2202.
de Lago et al. 2012. Cannabinoids ameliorate disease progression in a model of multiple sclerosis in mice, acting preferentially through CB(1) receptor-mediated anti-inflammatory effects. Neuropharmacology
Appendino et al. 2008. Antibacterial cannabinoids from Cannabis sativa: a structure study. Journal of Natural Products 71: 1427-1430.
University of Pittsburgh Medical Center Press Release. May 21, 2006. “Marijuana-derived drug suppresses bladder pain in animal models.”
Cecilia Hillard. 2000. Endocannabinoids and vascular function. Journal of Pharmacology and Experimental Therapeutics. 294: 27-32.
Kunos et al. 2000. Endocannabinoids as cardiovascular modulators. Chemistry and Physics of Lipids 108: 159-168.
Ribuot et al. 2005. Cardiac and vascular effects of cannabinoids: toward a therapeutic use? Annales de Cardiologie et d'Angeiologie (France) 54: 89-96.
Steven Karch. 2006. Cannabis and cardiotoxicity. Forensic Science, Medicine, and Pathology. 2: 13-18.
Steffens and Mach. 2006. Towards a therapeutic use of selective CB2 cannabinoid receptor ligands for atherosclerosis. Future Cardiology 2: 49-53.
Steffens et al. 2005. Low dose oral cannabinoid therapy reduces progression of atherosclerosis in mice. Nature 434: 782-786.
Pacher et al. 2005. Blood pressure regulation by endocannabinoids and their receptors (PDF). Neuropharmacology 48: 1130-1138.
ranjo Grotenhermen. 2006. Clinical pharmacodynamics of cannabinoids. In Russo et al (Eds) Handbook of Cannabis Therapeutics. Binghampton, N.Y.: Haworth Press.
Batkai et al. 2004. Endocannabinoids acting at cannabinoid-1 receptors regulate cardiovascular function in hypertension. Circulation 110: 1996-220.
Francois Mach. 2006. New anti-inflammatory agents to reduce atherosclerosis. Archives of Physiology and Biochemistry 112: 130-137.
Luvone et al. 2009. Cannabidiol: a promising drug for neurodegenerative disorders? CNS Neuroscience & Therapeutics 15: 65-75.
Sagredo et al. 2012. Cannabinoids: Novel Medicines for the Treatment of Huntington's Disease. Recent Patents on CNS Drug Discovery 7: 41-48.
Molina et al. 2011. Cannabinoid administration attenuates the progression of simian immunodeficiency virus. AIDS Research and Human Retroviruses 27: 585-592.
Ramirez et al. 2013. Attenuation of HIV-1 replication in macrophages by cannabinoid receptor 2 agonists. Journal of Leukocyte Biology 93: 801-810.
Riggs et al. 2012. A pilot study of the effects of cannabis on appetite hormones in HIV-infected adult men. Brain Research 1431: 46-52.
Gabbey et al. 2005. Endocannabinoids and liver disease—review. Liver International 25: 921-926.
Lavon et al. 2003. A novel synthetic cannabinoid derivative inhibits inflammatory liver damage via negative cytokine regulation. Molecular Pharmacology 64: 1334-1344.
Schnelle et al. 1999. Results of a standardized survey on the medical use of cannabis products in the German-speaking area. Forschende Komplementarmedizin (Germany) 3: 28-36.
Sylvestre et al. 2006. Cannabis use improves retention and virological outcomes in patients treated for hepatitis C. European Journal of Gastroenterology & Hepatology. 18: 1057-1063.
Marcu et al. 2010. Cannabidiol enhances the inhibitory effects of delta9-tetrahydrocannabinol on human glioblastoma cell proliferation and survival. Molecular Cancer Therapeutics 9: 180-189.
Preet et al. 2008. Delta9-Tetrahydrocannabinol inhibits epithelial growth factor-induced lung cancer cell migration in vitro as well as its growth and metastasis in vivo. Oncogene 10: 339-346.
Manuel Guzman. 2003. Cannabinoids: potential anticancer agents (PDF). Nature Reviews Cancer 3: 745-755.
Baek et al. 1998. Antitumor activity of cannabigerol against human oral epitheloid carcinoma cells. Archives of Pharmacal Research: 21: 353-356.
Carracedo et al. 2006. Cannabinoids induce apoptosis of pancreatic tumor cells via endoplasmic reticulum stress-related genes. Cancer Research 66: 6748-6755.
Michalski et al. 2008. Cannabinoids in pancreatic cancer: correlation with survival and pain. International Journal of Cancer 122: 742-750.
Ramer and Hinz. 2008. Inhibition of cancer cell invasion by cannabinoids via increased cell expression of tissue inhibitor of matrix metalloproteinases-1. Journal of the National Cancer Institute 100: 59 69.
Whyte et al. 2010. Cannabinoids inhibit cellular respiration of human oral cancer cells. Pharmacology 85: 328-335.
Leelawat et al. 2010. The dual effects of delta(9)-tetrahydrocannabinol on cholangiocarcinoma cells: anti-invasion activity at low concentration and apoptosis induction at high concentration. Cancer Investigation 28: 357-363.
Gustafsson et al. 2006. Cannabinoid receptor-mediated apoptosis induced by R(+)-methanandamide and Win55,212 is associated with ceramide accumulation and p38 activation in mantle cell lymphoma. Molecular Pharmacology 70: 1612-1620.
Gustafsson et al. 2008. Expression of cannabinoid receptors type 1 and type 2 in non-Hodgkin lymphoma: Growth inhibition by receptor activation. International Journal of Cancer 123: 1025-1033.
Natalya Kogan. 2005. Cannabinoids and cancer. Mini-Reviews in Medicinal Chemistry 5: 941-952.
Scott et al. 2013. Enhancing the activity of cannabidiol and other cannabinoids in vitro through modifications to drug combinations and treatment schedules 33: 4373-4380.
Aviello et al. 2012. Chemopreventive effect of the non-psychotropic phytocannabinoid cannabidiol on experimental colon cancer. Journal of Molecular Medicine.
Ruiz et al. 1999. Delta-9-tetrahydrocannabinol induces apoptosis in human prostate PC-3 cells via a receptor-independent mechanism. FEBS Letters 458: 400-404.
Mimeault et al. 2003. Anti-proliferative and apoptotic effects of anandamide in human prostatic cancer cell lines. Prostate 56: 1-12.
Cafferal et al. 2010. Cannabinoids reduce ErbB2-driven breast cancer progression through Akt inhibition. Molecular Cancer 9: 196.
Guzman et al. 2000. Anti-tumoral action of cannabinoids: involvement of sustained ceramide accumulation and extracellular signal-regulated kinase activation. Nature Medicine 6: 313-319.
Guzman et al. 2003. Inhibition of tumor angiogenesis by cannabinoids. The FASEB Journal 17: 529-531.
Naftali et al. 2011. Treatment of Crohn's disease with cannabis: an observational study. Journal of the Israeli Medical Association 13: 455-458.
Izzo and Coutts. 2005. Cannabinoids and the digestive tract. Handbook of Experimental Pharmacology 168: 573-598.
Izzo et al. 2009. Non-psychotropic plant cannabinoids: new therapeutic opportunities from an ancient herb. Trends in Pharmacological Sciences 30: 515-527.
Lal et al. 2011. Cannabis use among patients with inflammatory bowel disease. European Journal of Gastroenterology & Hepatology 23: 891-896.
David Secko. 2005. Analgesia through endogenous cannabinoids. CMAJ 173.
Wallace et al. 2007. Dose-dependent effects of smoked cannabis on capsaicin-induced pain and hyperalgesia in healthy volunteers. Anesthesiology 107:785-96.
Cox et al. 2007. Synergy between delta9-tetrahydrocannabinol and morphine in the arthritic rat. European Journal of Pharmacology 567: 125-130.
Ethan Russo. 2004. Clinical endocannabinoid deficiency (CECD): Can this concept explain therapeutic benefits of cannabis in migraine, fibromyalgia, irritable bowel syndrome and other treatment-resistant conditions? Neuroendocrinology Letters 25: 31-39.
Burns and Ineck. 2006. Cannabinoid analgesia as a potential new therapeutic option in the treatment of chronic pain. The Annals of Pharmacotherapy 40: 251-260.
Fiz et al. 2011. Cannabis use in patients with fibromyalgia: Effect on symptoms relief and health-related quality of life. PLoS One 6.
chley et al. 2006. Delta-9-THC based monotherapy in fibromyalgia patients on experimentally induced pain, axon reflex flare, and pain relief. Current Medical Research and Opinion 22: 1269-1276.
Dale Gieringer. 2001. Medical use of cannabis: experience in California. In: Grotenhermen and Russo (Eds). Cannabis and Cannabinoids: Pharmacology, Toxicology, and Therapeutic Potential. New York: Haworth Press: 153-170.
Porter and Jacobson. 2013. Report of a parnet survey of cannabidiol-enriched cannabis use in pediatric treatment-resistant epilepsy. Epilepsy & Behavior 29: 574-577.
Saundra Young, CNN.com. Aug. 7, 2013. “Marijuana stops child's severe seizures.”
abusch et al. 2004. Delta-9-tetrahydrocannabinol improves motor control in a patient with musician's dystonia (PDF). Movement Disorders 19: 990-991.
Fox et al. 2002. Randomised, double-blind, placebo-controlled trial to assess the potential of cannabinoid receptor stimulation in the treatment of dystonia. Movement Disorders 17: 145-149.
Richter et al. 2002. Effects of pharmacological manipulations of cannabinoid receptors on severe dystonia in a genetic model of paroxysmal dyskinesia. European Journal of Pharmacology 454: 145-151.
Consroe et al. 1986. Open label evaluation of cannabidiol in dystonic movement disorders. International Journal of Neuroscience 30: 277-282.
Richter et al. 1994. (+)-WIN 55212-2, a novel cannabinoid agonist, exerts antidystonic effects in mutant dystonic hamsters. European Journal of Pharmacology 264: 371-377.
Rajavashisth et al. 2012. Decreased prevalence of diabetes in marijuana users. BMJ Open 2
Rajesh et al. 2010. Cannabidiol attenuates cardiac dysfunction, oxidative stress, fibrosis, and inflammatory and cell death signaling pathways in diabetic cardiomyopathy. Journal of the American College of Cardiology 56: 2115-2125.
El-Remessy et al. 2006. Neuroprotective and blood-retinal barrier preserving effects of cannabidiol in experimental diabetes. American Journal of Pathology 168: 235-244.
Lu et al. 2006. The cannabinergic system as a target for anti-inflammatory therapies. Current Topics in Medicinal Chemistry 13: 1401-1426.
Croxford and Yamamura. 2005. Cannabinoids and the immune system: Potential for the treatment of inflammatory diseases. Journal of Neuroimmunology 166: 3-18.
Wilsey et al. 2013. Low-dose vaporized cannabis significantly improves neuropathic pain. The Journal of Pain 14: 136-148.
Comelli et al. 2008. Antihyperalgesic effect of a Cannabis sativa extract in a rat model of neuropathic pain. Phytotherapy Research 22: 1017-1024.
Ware et al. 2010. Smoked cannabis for chronic neuropathic pain: a randomized controlled trial. CMAJ 182: 694-701.
Raman et al. 2004. Amyotrophic lateral sclerosis: delayed disease progression in mice by treatment with a cannabinoid. Amyotrophic Lateral Sclerosis & Other Motor Neuron Disorders 5: 33-39.
Carter et al. 2010. Cannabis and amyotrophic lateral sclerosis: hypothetical and practical applications, and a call for clinical trials. American Journal of Hospice & Palliative Medicine 27: 347-356.
Eubanks et al. 2006. A molecular link between the active component of marijuana and Alzheimer's disease pathology. Molecular Pharmaceutics 3: 773-777.
Marchalant et al. 2007. Anti-inflammatory property of the cannabinoid agonist WIN-55212-2 in a rodent model of chronic brain inflammation. Neuroscience 144: 1516-1522.
Campbell and Gowran. 2007. Alzheimer's disease; taking the edge off with cannabinoids? British Journal of Pharmacology 152: 655-662.
Ramirez et al. 2005. Prevention of Alzheimer's disease pathology by cannabinoids. The Journal of Neuroscience 25: 1904-1913.
Carley et al. 2002. Functional role for cannabinoids in respiratory stability during sleep. Sleep 25: 399-400.

Many medicinal uses for cannabinoids and cannabis extracts are effective wherein cannabinoids are absorbed by the body more efficiently to local therapeutic effect.

But what is needed is a composition of at least one medicament and emu oil that enables more effective delivery of the at least one medicament into the respiratory tract.

### FIELD OF THE INVENTION

The present composition enables increased efficiency of the delivery of medicaments into the respiratory tract whereat relief from diseases of the respiratory tract is readily effected and convalescence and remediation significantly assisted thereby. The present invention contemplates a method for increasing the efficiency of the delivery of medicaments into the respiratory tract by effecting a vapor comprising the medicament in combination with emu oil and thence enabling inhalation of the vapor into the respiratory tract. The emu oil speeds the transfer of the medicament in combination with the emu oil into the respiratory tract and surrounding tissue. While the disclosure herein contemplates an exemplary composition of emu oil and cannabinoids, it is readily appreciated by a person of ordinary skill in the art that other medicaments may be usable for transport having emu oil as a carrier to enable more efficient delivery into the respiratory tract and surrounding tissue.

### SUMMARY OF THE INVENTION

Diseases of the lungs and respiratory tract are difficult and problematic to treat. As the lungs deteriorate the ability to effect gaseous exchange also decreases. Thus delivering medicine into the lungs for treatment is often impeded by the very condition the medicine seeks to counter. Medicines are therefore often introduced into the body intravenously. However, targeting the respiratory tract directly is preferable because medicaments inhaled are more concentrated and contact targeted tissue directly. However, inhaling particles often inflames or irritates the respiratory tract, rending inhalation a less desirable procedure than intravenous introduction of the medicament. Use of a carrier, therefore, is desirable where the carrier suppresses inflammation of the respiratory tract and speeds absorption of a coagent directly into respiratory and pulmonary tissue.

Emu oil has been shown to rapidly infuse across animal cell walls and through the cytoplasm. Emu oil has anti-inflammatory therapeutic properties in and of itself, but it is also particularly effective as a delivery agent to enable more efficient penetration of a coagent to a targeted area or tissue in the human body. The present method for delivery of medicaments via vaporized emu oil carrier, therefore, contemplates a composition of at least one medicament in combination with emu oil, which composition subjected to vaporization enables inhalation by a patient for direct application to the respiratory tract.

The present method for delivery of medicaments via vaporized emu oil carrier, therefore, includes a composition of emu oil and at least one medicament. The composition is subjected to heating interior to a volume. Vaporization is effective to produce a vapor comprising airborne droplets of emu oil intermixed and interspersed with the at least one medicament. The vapor is collected toward a singular point fluidly connected to the volume whereby a patient is enabled inhalation of the vapor. Once introduced into the respiratory tract, the emu oil droplets speed absorption of the medicament into the surrounding respiratory and pulmonary tissue while suppressing inflammation of the respiratory tract in response to inhaling the particles.

The present method contemplates a combination of emu oil and cannabinoids. The cannabinoids are dispersed in the emu oil as coagent. The volatilized emu oil thus carries the cannabinoids into the respiratory tract when inhaled, and delivers the cannabinoids directly to targeted tissue to effect treatment thereat. The emu oil carrier enables more efficient and complete absorption of cannabanoids directly into pulmonary tissue while suppressing inflammation caused by inhaling the vapor. Treatment of the respiratory tract is therefore more effectiviely administered.

Thus has been broadly outlined the more important features of the present method for delivery of medicaments via vaporized emu oil carrier so that the detailed description thereof that follows may be better understood and in order that the present contribution to the art may be better appreciated.

Objects of the present method for delivery of medicaments via vaporized emu oil carrier, along with various novel features that characterize the invention are particularly pointed out in the claims forming a part of this disclosure. For better understanding of the method for delivery of medicaments via vaporized emu oil carrier, its operating advantages and specific objects attained by its uses, refer to the accompanying drawings and description.

### BRIEF DESCRIPTION OF THE DRAWINGS

### Figures

FIG. 1 is a flow diagram of example steps informing the instant method.

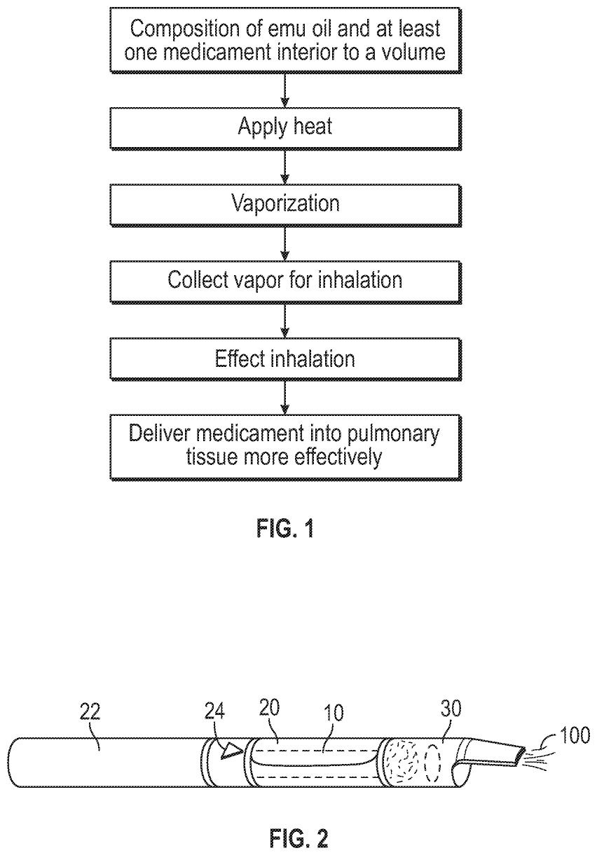

FIG. 2 is an in-use view of an example embodiment of demonstrating application of the method.

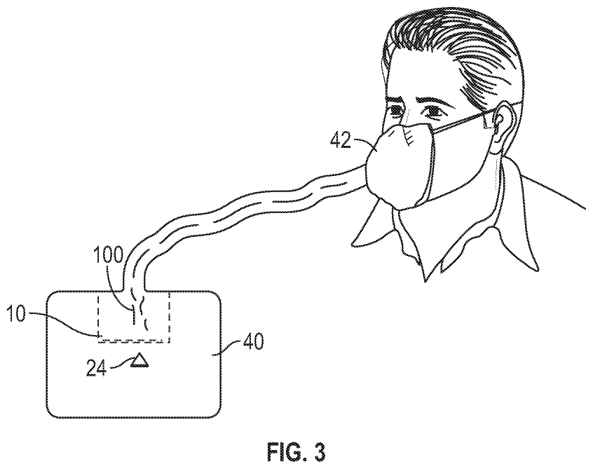

FIG. 3 is an in-use view of an example embodiment of demonstrating application of the method.

### DETAILED DESCRIPTION OF THE DRAWINGS

A method for delivery of medicaments via vaporized emu oil carrier is disclosed. A composition of emu oil and at least one medicament is disposed interior to a volume. Heat, in the range of 208° F. to 350° F. is applied to the composition to create a vapor. The vapor is maintained interior to the volume and then collected towards a singular point fluidly connected to the volume. The patient is thence enabled to inhale the vapor into the reparatory tract.

The emu oil comprising the composition serves as a carrier for the at least one medicament. The emu oil speeds absorption of said at least one medicament interior to the respiratory tract, increasing the speed and efficiency of penetration into and across the bronchi, bronchioles, and alveoli, penetrating surrounding pulmonary tissue. The at least one medicament is thereby more efficiently delivered into tissue for more effective treatment of disorders and diseases of the respiratory tract.

In an example embodiment contemplated herein, the at least one medicament includes cannabinoids.

FIG. 2 illustrates an example embodiment in-use by a patient. In this example embodiment 500 mg of cannabinoids are dissolved in 30 ml of emu oil. The composition is disposed in a cartridge 20 additional to a handheld vaporizer 22. The composition is subjected to approximately 208° F. to 350° F. and vaporization of the composition occurs for inhalation. Heating 24 may be effected automatically by action of a heating element activated when negative pressure, created across the mouthpiece 30 during inhalation, is sensed. Greater or lesser concentrations of the medicament dissolved, intermixed, suspended, or otherwise interspersed with the emu oil are contemplated as part of this disclosure.

FIG. 3 illustrates another example embodiment in-use by a patient. In this example the volume interior to which vaporization of the composition occurs is part of a tabletop vaporizer 40. The composition is delivered to the respiratory tract from the vaporizer 40 via a face mask 42 attachable to the face of a patient. Supine patients or hospitalized patients confined to a bed are thereby enabled to employ the instant method passively.

What is claimed is:

1. A method for the delivery of medicaments via a vaporized emu oil carrier for the treatment of respiratory disorders, said method comprising the steps of:
volatilizing a composition of emu oil and at least one medicament interior to a volume to create a vapor;
collecting the vapor towards a singular point fluidly connected to the volume; and
causing inhalation of the vapor by a patient;
wherein the emu oil serves as a carrier for the at least one medicament to speed absorption of the at least one medicament interior to the respiratory tract, into the lungs, across the bronchi, bronchioles, and alveoli, and into surrounding pulmonary tissue, whereby the at least one medicament is more efficiently transported for more effective treatment of disorders and diseases of the respiratory tract.

2. The method of claim 1 wherein a temperature of between 280 and 350° F. is applied to the composition to effect volatilization.

3. The method of claim 1 wherein the medicament includes cannabinoids.
